(19) United States
(12) Patent Application Publication
(10) Pub. No.: US 2020/0100696 A1
(43) Pub. Date: Apr. 2, 2020

(54) Physiological Parameter Monitor with a Cleat and an Equipment Module Removably Attachable to the Cleat

(71) Applicant: Welch Allyn, Inc, Skaneateles Falls, NY (US)

(72) Inventors: Timothy R. Fitch, Syracuse, NY (US); Scott Andrew Martin, Camillus, NY (US); Carlos Andres Suarez, Sracuse, NY (US); Thaddeus J. Wawro, Auburn, NY (US)

(21) Appl. No.: 16/148,113

(22) Filed: Oct. 1, 2018

(51) Int. Cl. A61B 5/0416; A61B 5/00

(52) U.S. Cl. CPC A61B 5/0416; A61B 5/04087; A61B 2562/0214; A61B 5/683

(57) ABSTRACT

A physiological monitor includes a cleat adapted to be securable to a subject such as a patient. The cleat includes an A electrode which includes an A electrode connector portion. The cleat also includes a B electrode which includes a B electrode connector portion. An equipment module is removably attached or removably attachable to the cleat in a transverse direction. The equipment module has an A module connector portion and a B module connector portion arranged such that the A and B module connector portions connect with the A and B electrode connector portions respectively when the equipment module is connected to the cleat thereby placing the cleat and equipment module in signal communication with each other.

### CROSS REFERENCE TO RELATED APPLICATIONS

This application claims priority to U.S. Provisional Application 62/621,232 filed on Jan. 24, 2018 and entitled “Physiological Parameter Monitor with a Cleat and an Equipment Module Removably Attachable to the Cleat”.

### TECHNICAL FIELD

The subject matter described herein relates to medical vital signs monitors and particularly to a modular monitor having a module which is wearable by a subject (e.g. a medical patient) and a detachable module which is detachable from the wearable module. When the patient participates in activities which might be harmful to components of the detachable module, the detachable module can be detached from the wearable module while the wearable module remains in place on the patient. The detachable module can be subsequently reattached to the wearable module.

### BACKGROUND

Wearable monitors for monitoring medical vital signs and other physiological parameters are advantageous because they can provide continuous monitoring of the vital signs of a subject, such as a hospital patient. Wearable monitors include an adhesive layer for securing the monitor to the patient. A caregiver presses the adhesive layer of the monitor against the patient's skin to secure the monitor to the patient's body.

One drawback of wearable monitors is the need to remove the monitor from the patient when the patient participates in activities that could cause damage to components of the monitor. Such activities include bathing, showering, and radiological procedures. At the conclusion of the activity it is desirable to resecure the wearable monitor to the patient. However the previous act of removing the monitor may have compromised the strength of the adhesive so that the monitor will no longer adhere reliably to the patient. Even if the monitor can be successfully reapplied to the patient, it is difficult to position the monitor exactly as it had been before removal. As a result the quality and/or consistency of the monitored signals may suffer. The monitor described herein overcomes at least these shortcomings of conventional vital signs monitors.

### SUMMARY

A physiological monitor comprises a cleat adapted to be securable to a subject, the cleat having an A electrode which includes an A electrode connector portion and a B electrode which includes a B electrode connector portion. The A and B connector portions are radially spaced from each other. The monitor also includes an equipment module removably attached or removably attachable to the cleat in a transverse direction. The module has an A module connector portion and a B module connector portion arranged such that the A and B module connector portions connect with the A and B electrode connector portions respectively when the equipment module is connected to the cleat.

### BRIEF DESCRIPTION OF THE DRAWINGS

The foregoing and other features of the various embodiments of the physiological monitor described herein will become more apparent from the following detailed description and the accompanying drawings in which:

FIG. 1 is a schematic cross sectional elevation view showing a cleat component and an equipment module component of the physiological monitor with the equipment module separated from the cleat and also showing A and B cleat electrodes, A and B connector portions of the electrodes, and A and B connector portions of the equipment module.

FIG. 2 is a view similar to FIG. 1 showing the equipment module attached to the cleat.

FIG. 3A is an elevation view showing the cleat of FIG. 1 with only the A electrode illustrated to assist the reader in identifying the features of the A electrode.

FIG. 3B is an elevation view showing the cleat of FIG. 1 with only the B electrode illustrated to assist the reader in identifying the features of the B electrode.

FIG. 4 is a perspective view of the cleat of FIG. 1.

FIG. 5 is an illustration showing the concepts of radial (R) and circumferential (C) in the context of a noncircular geometry (G).

### DETAILED DESCRIPTION

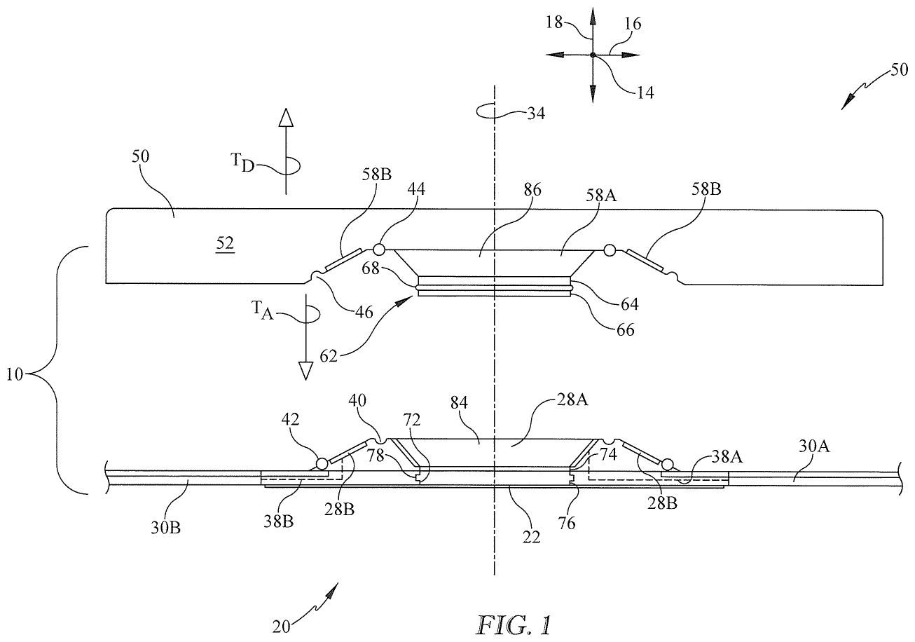

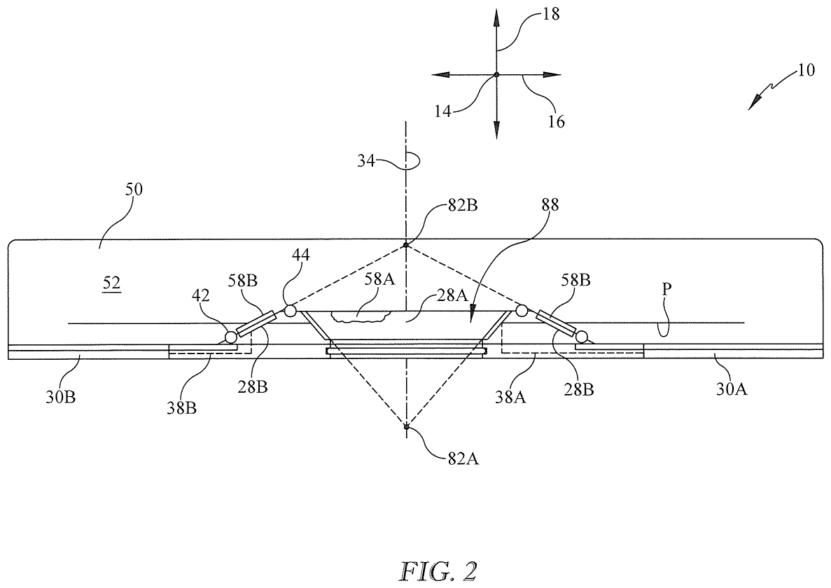

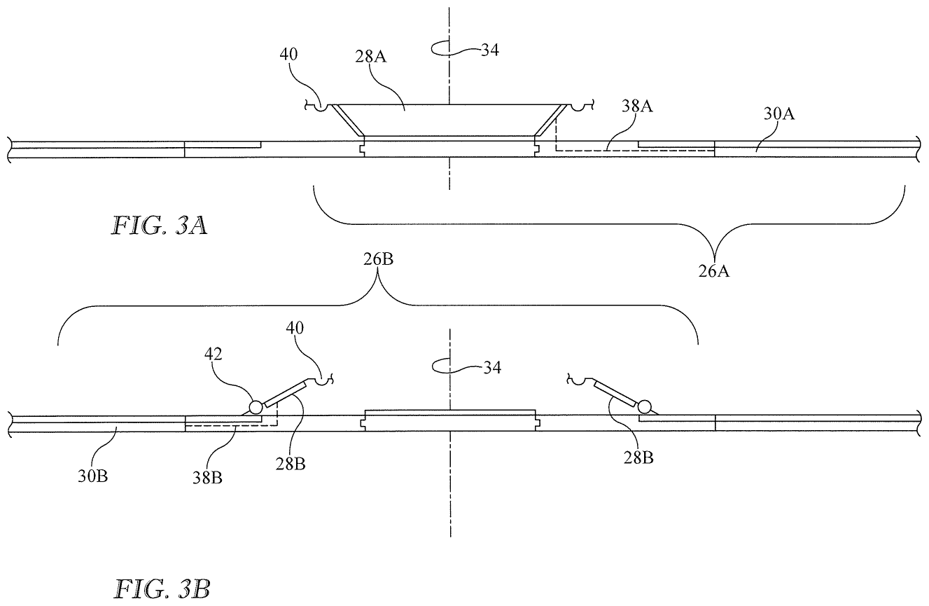

FIGS. 1 and 2 show a physiological monitor 10 in an assembled or connected state and a disassembled or disconnected state respectively and also show lateral, longitudinal, and transverse reference axes 14, 16, 18. The monitor includes a cleat 20 adapted to be securable to a subject, such as a patient. The illustrated cleat includes an adhesive layer 22 (shown only in FIG. 1) for securing the cleat to a patient. As seen best in FIGS. 3A and 3B, the cleat includes a pair of cleat electrodes 26A, 26B which are referred to herein as A and B electrodes. In one embodiment the electrodes are electrocardiogram (EKG) electrodes. In another embodiment the electrodes are excitation and sensing electrodes of the type employed in impedance electrocardgraphy.

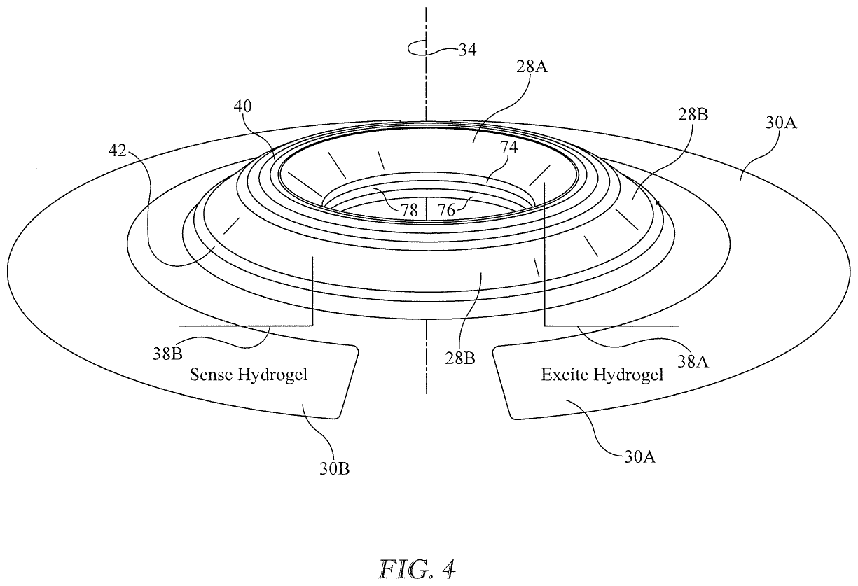

As seen best in FIGS. 3A and 4, A electrode 26A includes an A electrode connector portion 28A and an arcuate A contact portion 30A. Contact portion 30A is the portion of the A electrode intended to be in contact with the patient.

Referring additionally to FIG. 3B, B electrode 26B includes a B electrode connector portion 28B and an arcuate B contact portion 30B. Contact portion 30B is the portion of the B electrode intended to be in contact with the patient.

In embodiments in which the electrode contact portions 30A, 30B are EKG electrodes, the contact portions may hydrogel contact portions.

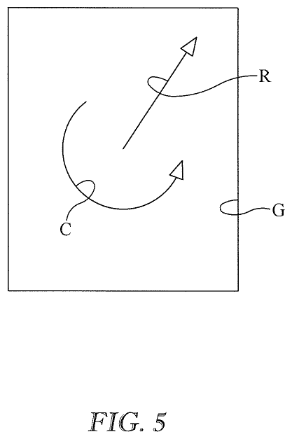

The A and B electrode connector portions 28A, 28B are radially spaced from each other. The A electrode connector portion 28A is radially inboard of the B electrode connector portion 28B. The A electrode contact portion 30A is radially spaced from and radially outboard of the A electrode connector portion 28A. The B electrode contact portion 30B is radially spaced from and radially outboard of the B electrode connector portion 28B. In this specification, the use of the term “radial” and variants thereof is not intended to limit the form of any component to a circular shape, such as that seen in FIG. 3. Instead, “radial” is used to describe spatial relationships relative to a reference point or axis, such as axis 34. The terms “inboard”, “outboard” and their variants are used to describe locations which are respectively closer to or more distant from a reference axis or reference point. The use of “circumferential” and its variants in this specification is also not intended to limit the form of any component to a circular shape. FIG. 5 shows the concepts of radial (R) and circumferential (C) in the context of a noncircular geometry (G).

A cleat O-ring seat 40 resides radially between the A and B electrode connector portions 28A, 28B. A cleat O-ring 42 resides radially between B connector portion 28B and contact portions 30A, 30B.

An A conductor element 38A connects the A contact portion 30A to the A connector portion 28A in order to convey signals between contact portion 30A and connector portion 28A. A B conductor element 38B connects the B contact portion 30B to the B connector portion 28B in order to convey signals between contact portion 30B and connector portion 28B. As illustrated, conductor elements 38A, 38B are considerably more localized than the connector and contact portions 28A, 28B, 30A, 30B, all of which have a substantial circumferential spread. Nevertheless, conductor elements 38A, 38B could be more circumferentially extensive than those illustrated.

The monitor also includes an equipment module 50 removably attached to or removably attachable to the cleat as described in more detail below. Equipment module 50 includes electronic components of the monitor housed in an equipment compartment 52. The electronic components may include at least a processor for processing data signals from electrodes 26A, 26B and from one or more sensors other than the electrodes (described below). The compartment may also house one or more amplifiers, one or more filters to amplify and de-noise the sensor and electrode signals, and a transceiver to provides communication with remote devices such as information displays and user controls. The compartment may also house a battery. Further examples of equipment modules, cleats, and their architectures may be found in U.S. provisional patent applications 62/588,598 filed on Nov. 20, 2017, 62/592,602 filed on Nov. 30, 2017, 62/618,772 filed on Jan. 18, 2018, and 62/607,646 filed on Dec. 19, 2017. The contents of the foregoing applications are incorporated herein by reference.

Equipment module 50 has an A module connector portion 58A and a B module connector portion 58B. The A module connector portion 58A is radially inboard of the B module connector portion 58B. As a result the A and B module connector portions 58A, 58B connect respectively with the A and B electrode connector portions 28A, 28B of the cleat when the equipment module is connected to the cleat as in FIG. 2. In the illustrated embodiment the A module connector portion 58A and the A electrode connector portion 28A are adapted to mate or connect with each other by being in mutual contact. The mutual contact establishes an A signal connection and resultant signal communication between the cleat and the equipment module, but does not establish a mechanical connection between the cleat and the equipment module. Likewise the B module connector portion 58B and the B electrode connector portion 28B are adapted to connect with each other by being in mutual contact thereby establishing a B signal connection and resultant signal communication, but not a mechanical connection, between the cleat and the equipment module. Mechanical connection is provided by mechanical connector elements described below.

An equipment module O-ring 44 resides radially between module connector portions 58A, 58B. An equipment module O-ring seat 46 resides radially outboard of B module connector portion 58B. When the equipment module is connected to the cleat as seen in FIG. 2, cleat O-ring 42 fits into module O-ring seat 46, and module O-ring 44 fits into cleat O-ring seat 40.

Equipment module 50 includes a module mechanical connector element 62 which extends transversely from the A module connector portion 58A. The illustrated connector element 62 comprises two cylindrical sections 64, 66 and an intervening ring 68 whose diameter is greater than that of the cylindrical sections.

Cleat 20 includes a cleat mechanical connector element 72 which extends transversely from A electrode connector portion 28A. The illustrated connector element 72 comprises two cylindrical sections 74, 76 and an intervening groove 78 with a diameter greater than that of the cylindrical sections. The module and cleat mechanical connector elements are adapted to mate with each other to affect a mechanical connection between cleat 20 and equipment module 50. In particular ring 68 snaps into groove 78 to affect the mechanical connection. The ring is also separable from the groove so that the equipment module can be detached from the cleat. As noted earlier in this specification, the fact that the equipment module is attachable to and detachable from cleat allows the equipment module to be temporarily removed from the cleat when the patient participates in activities that could cause damage to components of the equipment module. The equipment module can be subsequently re-attached to the cleat.

In the illustrated embodiment the mechanical connection between the cleat and the equipment module is distinct from the A signal connection defined by the A connector portions 28A, 58A of the cleat electrodes and the equipment module and is also distinct from the B signal connection defined by the B connector portions 28B, 58B of the cleat electrodes and the equipment module.

When the cleat and equipment module are connected to each other as seen in FIG. 2, The A and B electrode contact portions 30A, 30B are radially outboard of both the electrode A and B connector portions 28A, 28B and the A and B equipment module connector portions 58A, 58B.

In the specific embodiment shown in the drawings the A and B electrode connector portions 28A, 28B are circumferentially complete, i.e. they extend circumferentially 360 degrees to define an A signal connector ring 28A and a B signal connector ring 28B which circumscribes the A signal connector ring. The A cleat contact portion 30A is radially spaced from and is in signal communication, by way of conductor 38A, with the A cleat signal connector ring 28A. The B cleat contact portion 30B is radially spaced from and is in signal communication by way of conductor 38B, with the B cleat signal connector ring 28B. The A and B module connector portions 58A, 58B are also complete signal connector rings arranged so that the B module signal connector ring 58B circumscribes the A module signal connector ring 58A.

Referring to FIG. 2, the A cleat signal connector ring 28A and the A module signal connector ring 58A have similar frustoconical profiles having a virtual vertex 82A. The B cleat signal connector ring 28B and the B module signal connector ring 58B also have similar frustoconical profiles having a virtual vertex 82B. (In the illustration the conical profiles are represented by the interface between rings 28A and 28B and the interface between rings 58A and 58B, hence the presence of only a single apex on each side of the monitor.) “Similar” is used in the geometric sense of having the same shape. When the equipment module is connected to the cleat the apexes 82A, 82B are transversely separated from each other on opposite sides of center plane P. The A module signal connector ring 58A nests radially inside the A cleat signal connector ring 28A, and the B cleat signal connector ring 28B nests radially inside the B module connector ring 58B.

As seen in FIG. 1, cleat 20 includes a cleat sensor opening 84 radially inboard of cleat A electrode connector portion 28A. Equipment module 50 includes a module sensor opening 86 radially inboard of the A module connector portion 58A. When the cleat is assembled to the module as seen in FIG. 2, connector portions 84, 86 define a monitor sensor opening 88. In the specific embodiment in which the A module connector portion is a full ring 58A, the ring is the border of opening 88. The sensor opening is provided to accommodate sensors other than the electrodes 26A, 26B. Examples of such sensors include photoplethysmogram (PPG) sensors, phonocardiogram (PCG) sensors, and oxygen saturation (SpO2) sensors.

The monitor is in the assembled state of FIG. 2 when it is being used to monitor the physiological signs of a patient. The cleat and equipment module are in signal communication with each other by way of an A signal connection established by the A electrode connector portion 28A and the A module connector portion 58A and by the B electrode connector portion 28B and the B module connector portion 58B. The equipment module 50 is detachable from the cleat 20 in the transverse direction, as indicated by arrow T_{D} in FIG. 1, so that the patient may participate in activities which might be harmful to components of the detachable equipment module. When the activity is finished the equipment module can be reconnected to the cleat in direction T_{A} to reproduce the assembled state of FIG. 1.

Although this disclosure refers to specific embodiments, it will be understood by those skilled in the art that various changes in form and detail may be made without departing from the subject matter set forth in the accompanying claims.

We claim:

1. A physiological monitor comprising:
a cleat adapted to be securable to a subject, the cleat having an A electrode which includes an A electrode connector portion and a B electrode which includes a B electrode connector portion, the A and B connector portions being radially spaced from each other;
an equipment module removably attached or removably attachable to the cleat in a transverse direction, the module having an A module connector portion and a B module connector portion arranged such that the A and B module connector portions connect with the A and B electrode connector portions respectively when the equipment module is connected to the cleat.

2. The monitor of claim 1 wherein the A and B electrodes each include respective A and B contact portions adapted to contact the subject.

3. The monitor of claim 1 wherein the A and B electrodes are A and B EKG electrodes.

4. The monitor of claim 4 wherein one of the A and B electrodes is an excite electrode and the other is a sense electrode.

5. The monitor of claim 1 wherein the A and B electrode connector portions are circumferentially complete.

6. The monitor of claim 5 wherein the cleat includes a sensor opening radially inboard of the A electrode connector portion.

7. The monitor of claim 6 wherein the A electrode connector portion is a ring.

8. The monitor of claim 1 wherein:
the A electrode connector portion and the A module connector portion a define an A signal connection between the cleat and the module;
the B electrode connector portion and the B module connector portion define a B signal connection between the cleat and the module;
the cleat includes a cleat mechanical connector element;
the equipment module includes a module mechanical connector element; and
the cleat and module mechanical connector elements are adapted to mate with each other to affect a mechanical connection between the cleat and the equipment module which is distinct from the signal connections.

9. A physiological monitor comprising:
a radially extending cleat having:
an A electrode comprised of an A electrode connector portion and an A contact portion which is radially outboard of the A electrode connector portion; and
a B electrode comprised of a B electrode connector portion and a B contact portion which is radially outboard of the B electrode connector portion; and
an equipment module having:
an A module connector portion; and
a B module connector portion;
wherein the A module connector portion and the A electrode connector portion are adapted to contact each other, and the B module connector portion and the B electrode connector portion are adapted to contact each other.

10. The monitor of claim 9 wherein when the cleat and module are connected to each other the A electrode connector portion is radially inboard of the B electrode connector portion, and the A module connector portion is radially inboard of the B module connector portion.

11. The monitor of claim 10 wherein when the cleat and module are connected to each other the A and B electrode contact portions are radially outboard of both the electrode A and B connector portions and the A and B equipment module connector portions.

12. The monitor of claim 9 wherein:
the A electrode connector portion mates with the A module connector portion to define an A signal connection between the cleat and the module;
the B electrode connector portion mates with the B module connector portion to define a B signal connection between the cleat and the module;
the cleat includes a cleat mechanical connector element;
the equipment module includes a module mechanical connector element; and
the cleat and module mechanical connector elements are adapted to mate with each other to affect a mechanical connection between the cleat and the equipment module which is distinct from the signal connections.

13. A vital signs monitor comprising:
a cleat having:
an A cleat signal connector ring;
a B cleat signal connector ring circumscribing the A cleat signal connector ring;
an A cleat contact element radially spaced from and in signal communication with the A cleat signal connector ring;
a B cleat contact element radially spaced from and in signal communication with the B cleat signal connector ring;
a cleat mechanical connector element; and
an equipment module having:
an A module signal connector ring;
a B module signal connector ring circumscribing the A module signal connector ring;
a module mechanical connector element;
wherein the cleat and equipment module are connectable to an disconnectable from each other in a transverse direction.

14. The monitor of claim 13 wherein when the cleat and module are connected to each other:
the A cleat signal connector ring is in contact with the A module signal connector ring thereby establishing an A signal connection between the cleat and the module;
the B cleat signal connector ring is in contact with the B module signal connector ring thereby establishing a B signal connection between the cleat and the module; and
the cleat mechanical connector element and the module mechanical connector element are mated with each other to establish a mechanical connection between the cleat and the module.

15. The monitor of claim 13 wherein the A cleat signal connector ring and the A module signal connector ring have similar frustoconical profiles.

16. The monitor of claim 13 wherein the B cleat signal connector ring and the B module signal connector ring have similar frustoconical profiles.

17. The monitor of claim 13 wherein:
the A cleat signal connector ring and the A module signal connector ring have similar frustoconical profiles having A apexes;
the B cleat signal connector ring and the B module signal connector ring have similar frustoconical profiles having B apexes; and
when the cleat and module are connected to each other the A apexes lie on one side of a laterally and longitudinally extending centerplane and the B apexes lie on the other side of the laterally and longitudinally extending center plane.

18. The monitor of claim 17 wherein when the cleat and the module are connected to each other:
the A module signal connector ring nests radially inside the A cleat signal connector ring; and
the B cleat signal connector ring nests radially inside the B module connector ring.

19. The module of claim 13 wherein the A and B cleat contact elements have an arcuate profile.
